(19) United States
(12) Patent Application Publication
(10) Pub. No.: US 2011/0003847 A1
(43) Pub. Date: Jan. 6, 2011

(54) Prasugrel Salts with Improved Properties

(75) Inventor: Karlheinz Doser, Buchholz (DE)

Correspondence Address: SWANSON & BRATSCHUN, L.L.C., 8210 SOUTHPARK TERRACE, LITTLETON, CO, 80120, US

(73) Assignee: HELM AG, Hamburg (DE)

(21) Appl. No.: 12/865,887

(22) PCT Filed: Jan. 27, 2009
(86) PCT No.: PCT/EP09/50871

§ 371 Date: Aug. 5, 2010

Related U.S. Application Data
(60) Provisional application No. 61/026,492, filed on Feb. 6, 2008.
(60) Provisional application No. 61/113,800, filed on Nov. 12, 2008.

(51) Int. Cl. A61K 31/4365; C07D 471/04; A61P 9/00

(52) U.S. Cl. 514/301; 546/114

(57) ABSTRACT

Acid addition salts of 2-acetoxy-5-(α-cyclopropylcarbonyl-2-fluorobenzyl)-4,5,6,7-tetrahydrothieno[3,2-c]pyridine with sulfuric acid or sulfonic acids, pharmaceutical compositions comprising the same and processes for the production thereof. The acid addition salts have a low toxicity.

### FIELD OF THE INVENTION

This invention relates to new salts of prasugrel with improved physical and pharmaceutical properties and an improved toxico-logical profile.

### BACKGROUND OF THE INVENTION

Prasugrel is a thienopyridine compound of formula I currently undergoing clinical development of Phase III for the treatment of thrombosis and related diseases.

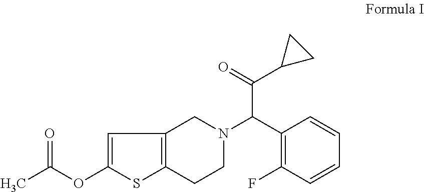

The chemical name of prasugrel is 2-acetoxy-5-(α-cyclopropylcarbonyl-2-fluorobenzyl)-4,5,6,7-tetrahydrothieno[3,2-c]pyridine. This compound has an asymmetric carbon in its molecule (C-7) and thus exists in the form of two enantiomers. Prasugrel is a base and forms acid addition salts with organic and inorganic acids.

The preparation of prasugrel and other tetrahydrothienopyridine derivatives as well the use thereof for inhibiting blood platelet aggregation and for the treatment and prophylaxis of thrombosis and embolism is described in EP 0 542 411 B1.

EP 0 785 205 B1 discloses 2-silyloxy-4,5,6,7-tetrahydrothieno[3,2-c]pyridines which are said to be useful as intermediates for synthesizing tetrahydrothienopyridines such as prasugrel.

U.S. Pat. No. 6,693,115 describes the preparation of acid addition salts of prasugrel with hydrochloric acid and maleic acid. The hydrochloride is preferred. These salts are said to exhibit excellent oral absorption, metabolization into the active compound, low toxicity and excellent storage and handling stability. The salts are reported to take up water and to form hydrates.

According to WO 2006/135605 the HCl and maleate salts of prasugrel have an improved stability profile when compared to other salts and the free base. Nevertheless, prolonged exposure of prasugrel-HCl to air and moisture results in degradation. Therefore, packaging in air and moisture impervious gas-inerted blister packages is recommended.

Furthermore hydrochloric acid salts are generally known to be corrosive, and maleate salts are susceptible to side reactions during storage (P. H. Stahl, C. G. Wermuth (Eds.), Handbook of Pharmaceutical Salts, Wiley-VCH, Weinheim, 2002). Therefore, there exists a need for prasugrel salts and formulations with improved stability.

### SUMMARY OF THE INVENTION

The present invention provides new acid addition salts of 2-acetoxy-5-(α-cyclopropylcarbonyl-2-fluorobenzyl)-4,5,6,7-tetrahydrothieno[3,2-c]pyridine (prasugrel) which exhibit improved physical and pharmaceutical properties and which have an improved toxicological profile. In particular the invention relates to prasugrel addition salts with a sulfur containing acid such as sulfuric acid and sulfonic acids, preferably sulfonic acids and more preferably alkylsulfonic acids or arylsulfonic acid such as methanesulfonic acid, benzene sulfonic acid, p-toluolsulfonic acid, 2-naphthalenesulfonic acid and 1,5 naphthalenedisulfonic acid. The invention also relates to pharmaceutical compositions comprising a pharmaceutically effective amount of one or more acid additions salts of prasugrel with an a sulfuric acid or sulfonic acid as well as methods for treating cardiovascular diseases comprising administering to a patient a formulation comprising such a composition.

### DETAILED DESCRIPTION OF THE INVENTION

The present invention relates to acid addition salts of 2-acetoxy-5-(α-cyclopropylcarbonyl-2-fluorobenzyl]-4,5,6,7-tetrahydrothieno[3,2-c]pyridine with sulfur containing acids such as sulfuric acid and preferably sulfonic acids, more preferably alkylsulfonic acids or arylsulphonic acids and relates to medicaments containing said acid addition salts of prasugrel.

The acid moiety of acid addition salts of 2-acetoxy-5-(α-cyclopropylcarbonyl-2-fluorobenzyl)-4,5,6,7-tetrahydrothieno-[3,2-c]pyridine is preferably a sulfonic acid, more preferably methanesulfonic acid, ethanesulfonic acid, ethane-1,2-disulfonic acid, 2-hydroxyethanesulfonic acid, benzene sulfonic acid, p-toluenesulfonic acid, 2-naphthalenesulfonic acid, naphthalene-1,5-disulfonic acid. Addition salts of unsubstituted arylsulfonic acid are particularly preferred, in particular the addition salts with benzene sulfonic acid or 2-naphthalenesulfonic acid. The resulting acid addition salts are the mesylate (methanesulfonic acid), esylate (ethanesulfonic acid), edysilate (ethane-1,2-disulfonic acid), isethionate (2-hydroxyethanesulfonic acid), besylate (benzene sulfonic acid), tosylate (p-toluenesulfonic acid), napsylate (2-naphthalinsulfonic acid), and the napadisylate (naphthalene-1,5-disulfonic acid).

2-Acetoxy-5-(α-cyclopropylcarbonyl-2-fluorobenzyl)-4,5,6,7-tetrahydrothieno[3,2-c]pyridine (prasugrel) has the following formula:

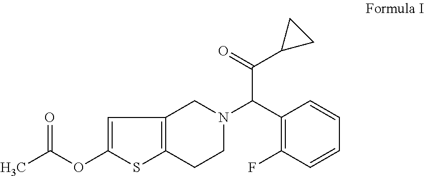

Acid addition salts of prasugrel have an asymmetric carbon in their molecule and in each compound two isomers having R and S configuration can exist. The present invention encompasses the individual isomers and mixtures of these isomers in optional proportions such as the racemate. An optically active isomer of acid addition salts of 2-acetoxy-5-(α-cyclopropylcarbonyl-2-fluorobenzyl)-4,5,6,7-tetrahydrothieno[3,2-c]pyridine of the present invention can be prepared using an optically active starting material or is isolated from a racemic mixture of synthetically prepared acid addition salts of 2-acetoxy-5-(α-cyclopropyl-carbonyl-2-fluorobenzyl)-4,5,6,7-tetrahydrothieno-[3,2-c]pyridine by a conventional optical resolution.

The acid addition salts of prasugrel according to the present invention can be prepared in the presence or absence of an inert solvent, preferably in an inert solvent, by addition of 2-acetoxy-5-(α-cyclopropylcarbonyl-2-fluorobenzyl)-4,5,6,7-tetrahydrothieno[3,2-c]pyridine to sulfuric acid or preferably a sulfonic acid, for instance by addition of 2-acetoxy-5-(α-cyclopropylcarbonyl-2-fluorobenzyl)-4,5,6,7-tetrahydrothieno-[3,2-c]pyridine to a sulfonic acid. 2-Acetoxy-5-(α-cyclopropylcarbonyl-2-fluorobenzyl)-4,5,6,7-tetrahydrothieno-[3,2-c]pyridine is preferably synthesized by a method described in EP 0 542 411 B1 or U.S. Pat. No. 6,693,115. The salts may also be prepared in the presence or absence of an inert solvent by dropwise addition of sulfuric acid or preferably a sulfonic acid to 2-acetoxy-5-(α-cyclopropylcarbonyl-2-fluorobenzyl)-4,5,6,7-tetrahydrothieno[3,2-c]pyridine. In this procedure seed crystals of said salt can be added, if available. The amount of acid to be added is from 0.1 equivalent to 2.0 equivalent, preferably from 0.5 to 1.5 and more preferably about 0.8 to 1.2 equivalent of acid.

The solvent used in the above reaction is not particularly restricted provided that it has no adverse effect on the reaction and it can dissolve the starting material to some extent. Examples of such solvents include aliphatic hydrocarbons such as hexane, cyclohexane, heptane, liguloin or petroleum ether; aromatic hydrocarbons such as benzene, toluene or xylene; halogenated hydrocarbons such as dichloromethane, chloroform, carbon tetrachloride, 1,2-dichloroethane, chlorobenzene or dichlorobenzene; ether derivatives such as diethyl ether, diisopropyl ether, tetrahydrofuran, dioxane, dimethoxyethane or di(ethyleneglycol)dimethyl ether; ketone derivatives such as acetone, methyl ethyl ketone or diethyl ketone; ester derivatives such as ethyl acetate, propyl acetate or butyl acetate; carboxylic acid derivatives such as acetic acid or propionic acid; or nitrile derivatives such as acetonitrile or propionitrile. Preferred solvents are nonhalogenated hydrocarbons, more preferred solvents are alcohols, ketones esters and ethers, still more preferred solvents are ketones. Acetone is the most preferred solvent

The reaction temperature will vary depending on the reagent, the solvent and the like, and usually is from −20° C. to 100° C., preferably from 0° C. to 70° C.

The reaction time will vary depending on the reagent, the solvent, the reaction temperature and the like, and usually is from 5 minutes to 10 hours, preferably 10 minutes to 5 hours.

The reaction is preferably carried out by addition of 2-acetoxy-5-(α-cyclopropylcarbonyl-2-fluorobenzyl)-4,5,6,7-tetrahydrothieno[3,2-c]pyridine to a solution of sulfonic acid in acetone between 0 and 70° C. followed by allowing to stand at said temperature for 30 minutes to 48 hours. Alternatively, the reaction is carried out by addition or drop-wise addition of the required amount of sulfonic acid to a solution of 2-acetoxy-5-(α-cyclopropylcarbonyl-2-fluorobenzyl)-4,5,6,7-tetrahydrothieno-[3,2-c]pyridine in acetone between 0° C. and 70° C. followed by allowing to stand at said temperature for 30 minutes to 48 hours. The crystallization of the salts can be accelerated by the addition of seed crystals and/or lowering the temperature to e.g. −20° C.

Prasugrel acid addition salts according to the present invention can also be prepared by exchanging the acid moiety of one salt by another acid moiety.

After the reaction, the acid addition salts of prasugrel can be isolated from the reaction mixture by conventional methods. For example, after the reaction, the resulting crystals are isolated by filtration to afford the desired product or the solvent of the reaction mixture is evaporated to afford the desired product. The product, if necessary, can be purified by recrystallization, reprecipitation or by chromatography.

It was surprisingly found by the present inventors that salts of prasugrel differ in their toxicity with regard to mammals. The salts of sulfonic acids showed lower toxicity than other salts. In particular the salt of benzene sulfonic acid (besylate) was found to have a very low toxicity. Although the exact reason for this low toxicity hat not yet been found, it is assumed that this effect is due to a different solubility of the salts. The toxicity and safety margin of prasugrel can be controlled by varying the solubility of its salts. Salts with lower solubility were found to be less toxic. It was particularly surprisingly found that sulfonic acid addition salts of prasugrel result in high blood levels of the active compound which are comparable to those of faster dissolving salts.

The preferred prasugrel salts according to the present invention are the addition salts with methanesulfonic acid (mesylate), ethanesulfonic acid (esylate), ethane-1,2-disulfonic acid (edisylate), 2-hydroxyethanesulfonic acid (isethonate), benzene sulfonic acid (besylate), p-toluenesulfonic acid (tosylate), 2-naphthalenesulfonic acid (napsylate), or naphthalene-1,5-disulfonic acid (napadisylate). Camphor-10-sulfonic acid (camsylate) is useful for the separation of the enantiomers. Addition salts with acidic ion exchange resins containing sulfonic acid groups are useful for controlled release formulations. Particularly preferred are the salts with benzene sulfonic acid (besylate), 2-naphthalenesulfonic acid (napsylate) and 1,5-naphthalenedisulfonic acid (napadisylate).

Moreover, the addition salts of prasugrel with sulfonic acids, and in particular prasugrel besylate, prasugrel napsylate and prasugrel napadisylate are not hygroscopic and not corrosive.

The acid addition salts of the present invention are preferably used in crystalline form. However, for some applications it may be advantageous to use the acid addition salts of prasugrel in non-crystalline form. For instance, non-crystalline forms of an active ingredient often have a higher solubility than the crystalline forms. The manufacturing of pharmaceutical formulations containing non-crystalline active ingredients is usually difficult. It was found by the present inventors that non-crystalline forms of the addition salts of the present invention can be prepared by coating the salt on particles as will be described below. These coated particles can than be directly used for the preparation of pharmaceutical formulations.

The prasugrel salts of the invention can contain solvent molecules such as water molecules or they can be solvent-free. Thus, the present invention also encompasses hydrates and solvates of the prasugrel salts.

The acid addition salts of prasugrel with sulfonic acids of the present invention exhibit excellent oral absorption, activity in inhibition of platelet aggregation, and excellent storage and handling stability. They are useful for the treatment and/or prevention of thrombosis and cardiovascular diseases such as acute coronary syndrome, cerebro vascular disease, high risk vascular disease, coronary occlusion, congestive heart failure, cardiac alternation, ventricular aneurysm, mural aneurysm, myocardial infarction, cardiac arrest, cardiac dysrhythmia, cardiac edema, cardiac dyspnea, cardiac failure, tachycardia, cardiac hemoptysis, cardiac incompetence, cardiac murmur, cardiac syncope, cardiac tamponade and peripheral vascular disease. The acid addition salts of prasugrel according to the present invention are particularly useful as a medicament for the inhibition of platelet aggregation The above medicaments are preferably for a mammal, more preferably a human.

The prasugrel acid addition salts of the present invention can be administered alone or in the form of pharmaceutical compositions comprising pharmaceutically acceptable excipients, diluents and the like, in various dosage forms such as tablets, capsules, granules, powders, syrups or the like for oral administration; and injections, suppositories or the like for parenteral administration. Pharmaceutical compositions for oral administration in particular tablets are preferred. The tablets may be film coated or uncoated.

Each of the above formulations can be prepared by well-known methods using additives for the formulation such as excipients, lubricants, glidants, binders, disintegrants, emulsifiers, stabilizers, corrigents, and diluents.

Examples of excipients include organic excipients, for example sugar derivatives such as lactose, sucrose, glucose, mannitol, xylitol or sorbitol; starch derivatives such as corn starch, potato starch, a starch, dextrin or cyclodextrin; cellulose derivatives such as microcrystalline cellulose; acacia; dextran; pullulan; and inorganic excipients; for example silicate derivatives such as light silicic acid anhydride, e.g. colloidal silica, precipitated silica and pyrogenic silica, synthetic aluminum silicate, calcium silicate, or magnesium aluminate metasilicate; phosphate derivatives such as calcium hydrogenphosphate; carbonate derivatives such as calcium carbonate; sulfate derivatives such as calcium sulfate, or the like.

Examples of lubricants include fatty acids such as stearic acid; metal stearate derivatives such as calcium stearate or magnesium stearate or esters of fatty acids; talc; waxes such as beeswax or spermaceti; boric acid; adipic acid; sulfate derivatives such as sodium sulfate; glycol; fumaric acid; sodium benzoate; DL-leucine; lauryl sulfate derivatives such as sodium lauryl sulfate or magnesium lauryl sulfate; silicic acid derivatives such as silicic anhydride or silicic acid hydrate; and starch derivatives as described in the excipients above.

Examples of binders include saccharose; gelatin; xylitol; dextrases; alginic acid and its derivatives; pectin; tragacanth; cellulose derivatives such as microcrystalline cellulose, (Na—) carboxymethyl cellulose, methyl cellulose, hydroxypropylcellulose, hydroxypropylmethyl cellulose, hydroxyethylcellulose, hydroxypropyl cellulose, ethylcellulose, ethyl hydroxyethyl cellulose, cellulose acetate; polyacrylic acid; polyacrylamide; polymethacrylate; polyvinylacetate; polyvinylpyrrolidon; vinylpyrrolidon-vinylacetate copolymer (Kollidon®); methylvinylether copolymers (Gantrez®); polyethylenglycols (Macrogol®); starches and modified starches, such as maltodextrine, sodium starch glycolate, sodium starch glycolate (Primojel®); or excipients as described in the excipients above.

Examples of disintegrants include cellulose derivatives such as lower-substituted hydroxypropylcellulose, car-boxymethylcellulose, calcium carboxymethylcellulose or internally cross-linked sodium carboxymethylcellulose; chemically modified starch or cellulose derivatives such as carboxymethylstarch or sodium carboxymethylstarch; cross-linked polyvinylpyrrolidine; and starch derivatives as described above; polysacharides such as guar gum, xanthan gum, cyclodextrines and ion exchange resins (e.g. Amberlite®).

Examples of emulsifiers include colloidal clay such as bentonite or veegum; metal hydroxides such as magnesium hydroxide or aluminum hydroxide; anionic surfactants such as sodium lauryl sulfate or calcium stearate; cationic surfactants such as benzalkonium chloride; non-ionic surfactants such as polyoxyethylene alkyl ether, polyoxyethyene sorbitan esters of fatty acids or sucrose esters of fatty acids.

Examples of stabilizers include para-hydroxybenzoic acid ester derivatives such as methylparaben or propylparaben; alcohol derivatives such as chlorobutanol, benzyl alcohol or phenethyl alcohol; benzalkonium chloride; phenol derivatives such as phenol or cresol; thimerosal; dehydroacetic acid or sorbic acid; complexing agents such as EDTA; antioxidants such as ascorbic acid or ascorbyl palmitate.

Examples of corrigents include sweeteners, souring agents, flavorings or the like which are conventionally used. The pH of the tablets can be controlled by adding acids such as ascorbic acid, citric acid, fumaric acid or alkaline compounds such as Ca-carbonate or Na-carbonate.

The pharmaceutical formulations can be film coated using common coating materials such as modified celluloses, polymethacrylates polyvinylpyrrolidones, polyvinylacetate phthalate or other materials known to those skilled in the art. Preferred coating materials are substances which will protect the active ingredient against moisture and/or air.

The specific dose of a compound of the present invention will be varied according to the severity of the patient's symptoms, age and the like. For oral administration the quantity of active ingredient in a unit dosage may be in the range of 0.1 mg to 1000 mg, preferably 1 mg to 500 mg, calculated as the free base. A unit dose for intravenous administration may be in the range of 0.01 mg to 500 mg, preferably 0.1 mg to 250 mg, of a compound of the present invention, calculated as the free base. The unit dose may be administered to a human adult from 1 to 7 times per a day for a period of from 1 to 7 days depending on the severity of the patient's symptoms.

The addition salts of prasugrel according to the present invention can also be used in combination with other active ingredients such as cardioprotective agents and anticoagulants such as acetylsalicylic acid (aspirin), heparins, Faktor X-inhibitors, fibrinogen-receptor antagonists, vitamin-K-antagonists, HMG-CoA-reductase inhibitors and other lipid lowering agents, COX-2 inhibitors, ACE-inhibitors, angiotensin-II-antagonists, NEP-inhibitors, Ca-antagonists, beta-blockers, sterine absorption inhibitors such as ezetimibe. Combinations with acetylsalicylic acid (aspirin) are preferred.

In order to minimise degradation, water and oxygen are preferably excluded during manufacturing and storage of the pharmaceutical compositions of the present invention. Compaction, dry granulation and dry compression are preferred procedures. In one preferred procedure the prasugrel salt and the excipients are blended, compacted, sieved and pressed to tablets. In another procedure the excipients are wet granulated and dried and the prasugrel salt is then mixed with the dried granulate. Most preferably the compositions of the present invention are prepared by a dry compression process. Alternatively, the prasugrel salts of the present invention can be coated on particles, e.g. by dissolving the desired prasugrel salt in a suitable solvent in the presence of dispersed carrier particles and removing the solvent, or by spraying the solution on carrier particles followed by drying of the particles.

The pharmaceutical compositions of the present invention can also have the form of solid solutions (dispersions). Preferred excipients for preparing solid solutions are eudragits, glycerol-behenate, glycerol-monooleate, glycerol-monostearate and glycerol-palmitostearate.

The acid addition salts of prasugrel according to the present invention are useful for the preparation of immediate release or controlled release preparations. The salts of the present invention are particularly suited for the preparation of solid immediate release formulations such as tablets, capsules and caplets.

Immediate release formulations (tablets, capsules caplets) contain 0 1 to 1000 mg, preferably 1 to 500 mg, and more preferably 5 to 60 mg prasugrel salt, calculated as the free base.

Immediate release pharmaceutical compositions of the present invention preferably comprise:

| Ingredient | amount |
|---|---|
| Prasugrel salt | 1-70 wt.-%, preferably 3-30 wt.-%; |
| Diluent | 20-95 wt.-%, preferably 40-80 wt.-%; |
| Glidant | 0.1-4 wt.-%, preferably 0.5-3 wt.-%; |
| Binder | 5-70 wt.-%, preferably 10-30 wt.-%; |
| Lubricant | 0.1-10 wt.-%, preferably 0.5-3 wt.-%. |
| Disintegrant | 0.1-30 wt.-%, preferably 0.5-20 wt.-% |

All percentages are based on the total weight of the composition.

Particularly preferred diluents are mannitol and/or lactose, a preferred glidant is silicon dioxide, a preferred binder is microcrystalline cellulose, preferred lubricants are stearic acid and stearic acid salts such as Mg-stearate. A particularly preferred disintegrant is croscarmellose sodium (e.g. Ac—Di—Sol®). Chemically, croscarmellose sodium is the sodium salt of a cross-linked, partly O-(carboxymethylated) cellulose.

Sustained release formulations can be prepared by use of excipients known to one of skill in the art such as swellable polymethacrylates, cellulose and starch derivatives as defined above. Preferred materials for preparing controlled release preparations are waxes or fats; glycerol monostearate, behenate, monooleate, palmitostearate; polymers such as Eudragit® and Ultramid®, polyethylene, polyvinylacetate, polymethacrylate, polyvinylchloride, ethyl cellulose, hydroxypropylmethyl cellulose, cellulose acetylphthalate; ion exchange resins such as Amberlite®.

The following examples and formulation examples are intended to further illustrate the present invention and are not intended to limit the scope of this invention.

### BRIEF DESCRIPTION OF THE FIGURES

FIG. 1 shows the ¹³C-NMR spectrum of prasugrel-2-napsylate in [D₆] DMSO.

FIG. 2 shows the ¹H-NMR spectrum of prasugrel-2-napsylate in [D₆] DMSO.

FIG. 3 shows the ¹³C-NMR spectrum of prasugrel ethanesulfonate (esylate) in [D₆] DMSO.

FIG. 4 shows the ¹H-NMR spectrum of prasugrel ethanesulfonate (esylate) in [D₆] DMSO.

FIG. 5 shows the ¹³C-NMR spectrum of prasugrel mesylate in CDCl₃.

FIG. 6 shows the ¹³C-NMR spectrum of prasugrel besylate in CDCl₃.

FIG. 7 shows the X-Ray diffractogram of prasugrel-1,5-dinapsylate.

FIG. 8 shows the X-Ray diffractogram of prasugrel-mesylate.

FIG. 9 shows the X-Ray diffractogram of prasugrel-1-napsylate.

FIG. 10 shows the X-Ray diffractogram of prasugrel-1,2-diesylate.

FIG. 11 shows the X-Ray diffractogram of prasugrel-besylate.

### EXAMPLES

### Example 1

### Preparation of 2-Acetoxy-5-(α-cyclopropylcarbonyl-2-fluorobenzyl)-4,5,6,7-tetrahydrothieno[3,2-c]pyridine (Prasugrel)

2-Acetoxy-5-(α-cyclopropylcarbonyl-2-fluorobenzyl)-4,5,6,7-tetrahydrothieno[3,2-c]pyridine is prepared by the method described in EP 0 785 205 B1.

To a mixture of 3.27 g of 5,6,7,7a-tetrahydro-4H-thieno-[3,2-c]pyridin-2-one-p-toluenesulfonate, 0.66 g of tri-ethylamine and 7 ml of methylene chloride is added 1.58 g of tert-butyl-dimethylchlorosilane, and the mixture is stirred at 25° C. for 25 hours to obtain a mixed solution. To the obtained mixed solution are added 2.02 g of triethylamine and 2.12 g of 2-fluoro-cyclopropylcarbonylbenzyl chloride, and the mixture is allowed to react under stirring at 40° C. for 12 hours.

After 20 ml of methylene chloride and 20 ml of 0.1 N-hydrochloric acid are added to the obtained reaction mixture, separation operation is carried out to obtain an organic layer. The obtained organic layer is washed with 20 ml of a 5% sodium hydrogen carbonate aqueous solution and 20 ml of water, dried over anhydrous magnesium sulfate and concentrated under reduced pressure to obtain a residue. To the obtained residue is added 15 ml of acetonitrile, and the mixture is cooled to 0° C. to obtain precipitated crystals. The obtained precipitated crystals are filtered and dried to obtain 2.24 g of 2-(tertbutyldimethylsilyloxy)-5-(α-cyclopropylcarbonyl-2-fluorobenzyl)-4,5,6,7-tetrahydrothieno[3,2-]pyridine.

Next, a mixture of 1.0 g of 2-(tert-butyldimethylsilyloxy)-5-(α-cyclopropylcarbonyl-2-fluorobenzyl)-4,5,6,7-tetrahydrothieno[3,2-c]pyridine, 0.46 g of triethylamine, 14 mg of 4-dimethylaminopyridine and 3 ml of acetonitrile is stirred at room temperature (20° C.) for 0.25 hour. Thereafter, 2 ml of an acetonitrile solution in which 0.46 g of acetic anhydride is dissolved is added dropwise to the mixture, and the resulting mixture is allowed to react under further stirring at room temperature (20° C.) for 1 hour.

To the obtained reaction mixture is added 3.3 ml of a 10 mM aqueous potassium dihydrogen phosphate solution, and the mixture is stirred at room temperature (20° C.) for 1 hour to obtain precipitated crystals. The obtained precipitated crystals are filtered and dried to obtain 0.76 g of 2-acetoxy-5-(α-cyclopropylcarbonyl-2-fluorobenzyl)-4,5,6,7-tetrahydrothieno-[3,2-c]pyridine. The purity of the product as determined by HPLC was 99.5%.

¹H-NMR (CDCl₃; standard: tetramethylsilane ; equipment Bruker DTX 200, 200.1 MHz): δ=0.76-1.15 (m; 4H), 2.17-2.34 (m; 1H), 2.26 (s; 3H), 2.58-3.05 (m; 4H), 3.57 (AB-system; 2H), 4,88 (s; 1H), 6,27 (s; 1H), 7,07-7,55 (m; 4H) ppm.

¹³C NMR (CDCl₃; standard: tetramethylsilane; equipment: Bruker DTX 200, 50,3 MHz): δ=11.98 (s), 12.58 (s), 18.79 (s), 21.09 (s), 25.44 (s), 48.91 (s), 50.96 (s), 72.04 (s), 112.39 (s), 116.28 (d; 22,9 Hz), 122.43 (d; 14,1 Hz), 124.9 (d, 2.9 Hz), 126.14 (s), 129.79 (s), 130.43 (d; 8,1 Hz), 131.07 (d; 2.9 Hz), 149.98 (s), 161.75 (d; 247.3 Hz), 168.14 (s), 208.04 (s) ppm.

### Example 2

### Preparation of Acid Addition Salts of Prasugrel with Sulfonic Acids (General Procedure)

One gram of prasugrel base in 15 ml acetone is added to a solution of 1 equivalent sulfonic acid in 15 ml acetone at 40° C. within 5 minutes with stirring. After completion of the addition the reaction mixture is stirred for an additional 2 hours at room temperature. The resulting crystals are isolated by filtration and dried at 60° C. under reduced pressure.

### Example 3

### Preparation of Acid Addition Salts of Prasugrel with Sulfonic Acids (General Procedure)

One gram of prasugrel base is dissolved in 20 ml ethanol and a solution of 1 equivalent sulfonic acid in 20 ml ethanol is added. The mixture is stirred for 30 minutes at 40° C. Then the solvent is removed under reduced pressure and the residue is dried in vacuum.

### Example 4

### Preparation of Immediate Release Pharmaceutical Compositions Containing Addition Salts of Prasugrel with Sulfonic Acids

Immediate release tablets are prepared by dry mixing the ingredients given in the following table and by dry compression of the obtained mixture.

| | amount |
|---|---|
| Ingredient | [wt.-%] |
| prasugrel besylate | 10 |
| mannitol | 65 |
| silicon dioxide | 1 |
| microcrystalline cellulose | 18 |
| Mg-stearate | 1 |
| corn starch | 5 |

### Example 5

### Preparation of Immediate Release Pharmaceutical Compositions Containing Addition Salts of Prasugrel with Sulfonic Acids

Immediate release tablets are prepared by dry mixing the ingredients given in the following table and by dry compression of the obtained mixture.

| | amount |
|---|---|
| Ingredient | [wt.-%] |
| prasugrel esylate | 2 |
| aspirin | 38 |
| mannitol | 33 |
| silicon dioxide (Aerosil) | 1 |
| microcrystalline cellulose | 20 |
| Mg-stearate | 1 |
| corn starch | 5 |

### Example 6

### Preparation of Sustained Release Pharmaceutical Compositions Containing Addition Salts of Prasugrel with Sulfonic Acids

Sustained release tablets are prepared by dry mixing the ingredients given in the following table and by dry compression of the obtained mixture.

| | amount |
|---|---|
| Ingredient | [wt.-%] |
| prasugrel napsylate | 10 |
| polyvinylpyrrolidon (PVP) | 2 |
| lactose | 30 |
| microcrystalline cellulose | 22 |
| HPMC | 25 |
| Na starch glycolate | 10 |
| Mg-stearate | 1 |

### Example 7

### Preparation of Sustained Release Pharmaceutical Compositions Containing Addition Salts of Prasugrel with Sulfonic Acids

Sustained release tablets are prepared by dry mixing the ingredients given in the following table and by dry compression of the obtained mixture.

| | amount |
|---|---|
| Ingredient | [wt.-%] |
| prasugrel besylate | 10 |
| lactose | 20 |
| glycerol behenate | 25 |
| PVP | 7 |
| Mg-stearate | 1 |
| sorbite | 37 |

### Example 8

### Preparation of Prasugrel-2-Napsylate

250 mg prasugrel (free base) were dissolved in 5 ml of methanol and the solution was stirred at room temperature. An equimolar amount of 2-naphthalene sulfonic acid dissolved in 2 ml methanol was added. The solvent was evaporated in a rotatory evaporator and dried over night in vacuum.

¹³C NMR ([D₆]DMSO; standard: tetramethylsilane; equipment: Bruker DTX 200, 50.3 MHz): δ=13.44 (s), 13.89 (s), 20.55 (s), 21.33 (s), 22.63 (s), 50.5 (broad), 70.1 (s), 112.78 (s), 116.01 (d; 14.1 Hz), 117.73 (d; 21.2 Hz), 124.63 (s), 124.79 (s), 124.99 (s), 125.07 (s), 126.73 (s), 127.18 (s), 127.35 (s), 128.25, 128.36, 129.29 (s), 133.01 (s), 133.16 (s), 133.64 (s), 134.59 (d; 8.5 Hz), 146.27 (s), 150.74 (s), 161.87 (d; 250.1 Hz), 168.51 (s), 202.84 (s) ppm.

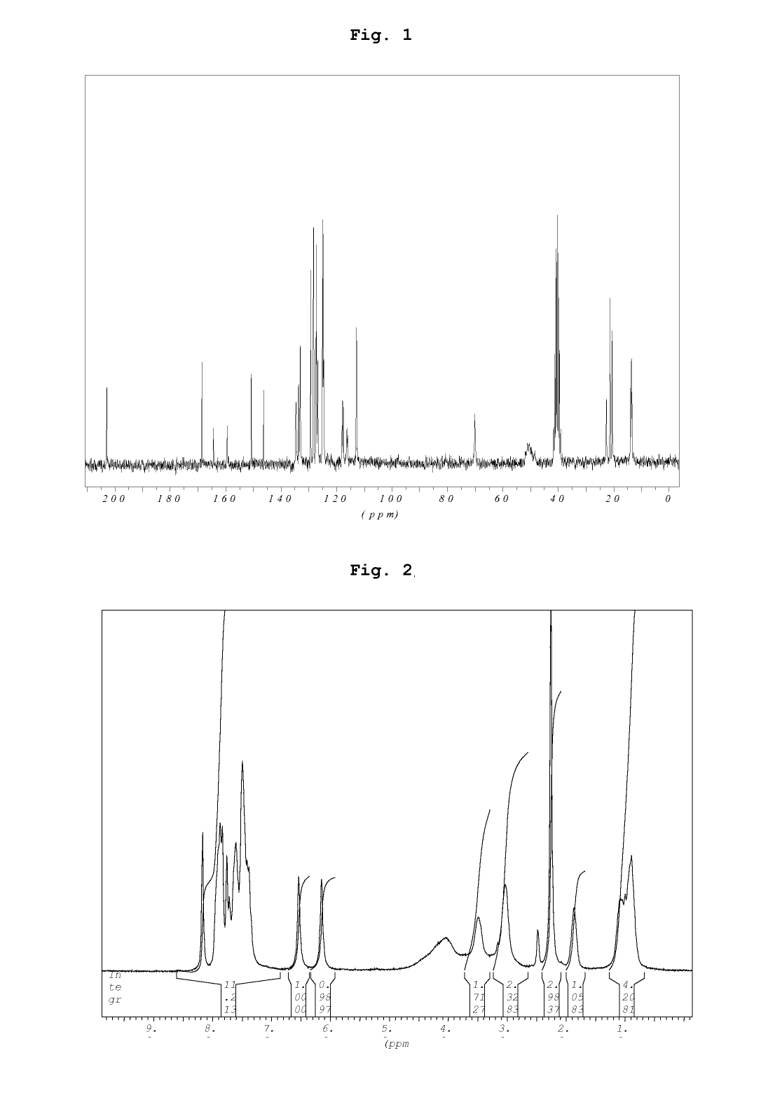

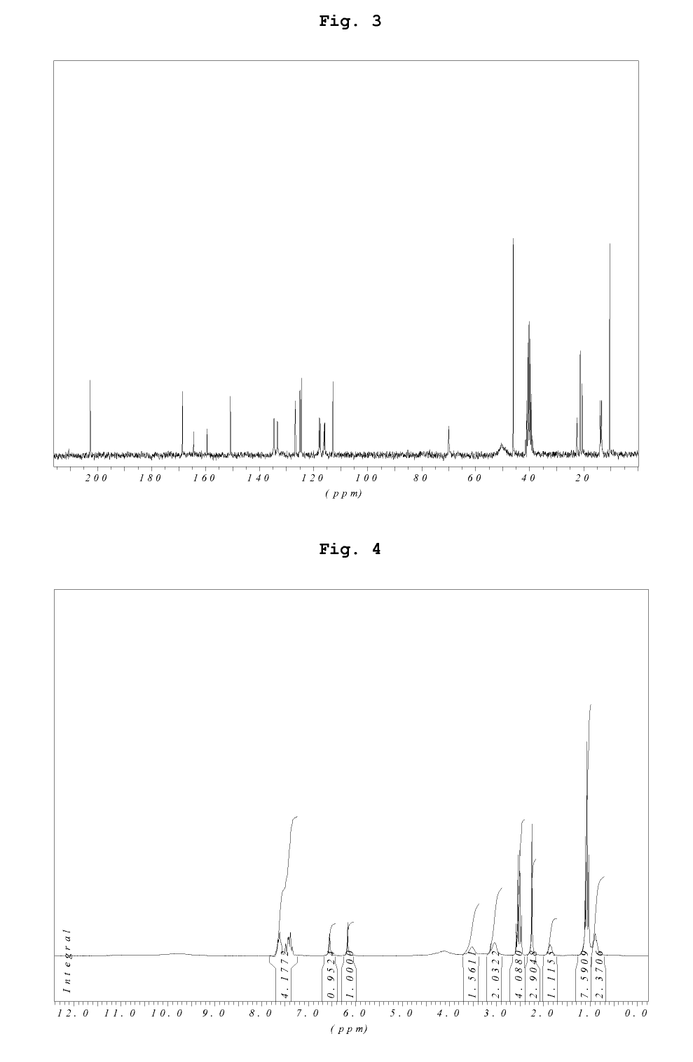

The ¹³C-NMR spectrum of prasugrel-2-napsylate is shown in FIG. 1, the ¹H-NMR spectrum in FIG. 2.

### Example 9

### Preparation of Prasugrel Ethanesulfonate (Esylate)

250 mg prasugrel (free base) were dissolved in 5 ml of methanol and the solution was stirred at room temperature. An equimolar amount of ethane sulfonic acid dissolved in 2 ml methanol was added. The solvent was evaporated in a rotatory evaporator and dried over night in vacuum.

¹³C NMR ([D₆]DMSO; standard: tetramethylsilane; equipment:

Bruker DTX 200, 50.3 MHz): δ=10.34 (s), 13.41 (s), 13.87 (s), 20.53 (s), 21.28 (s), 22.47(s), 46.09(s), 50.42 (broad), 69.91 (s), 112.78 (s), 116.20 (d; 14.1 Hz), 117.73 (d; 21.2 Hz), 124.48 (s), 125.05 (s), 126.76 (s), 133.3 (s), 134.65 (d; 8.5 Hz), 150.74 (s), 161.87 (d; 250.1 Hz), 168.51 (s), 202.65 (s) ppm.

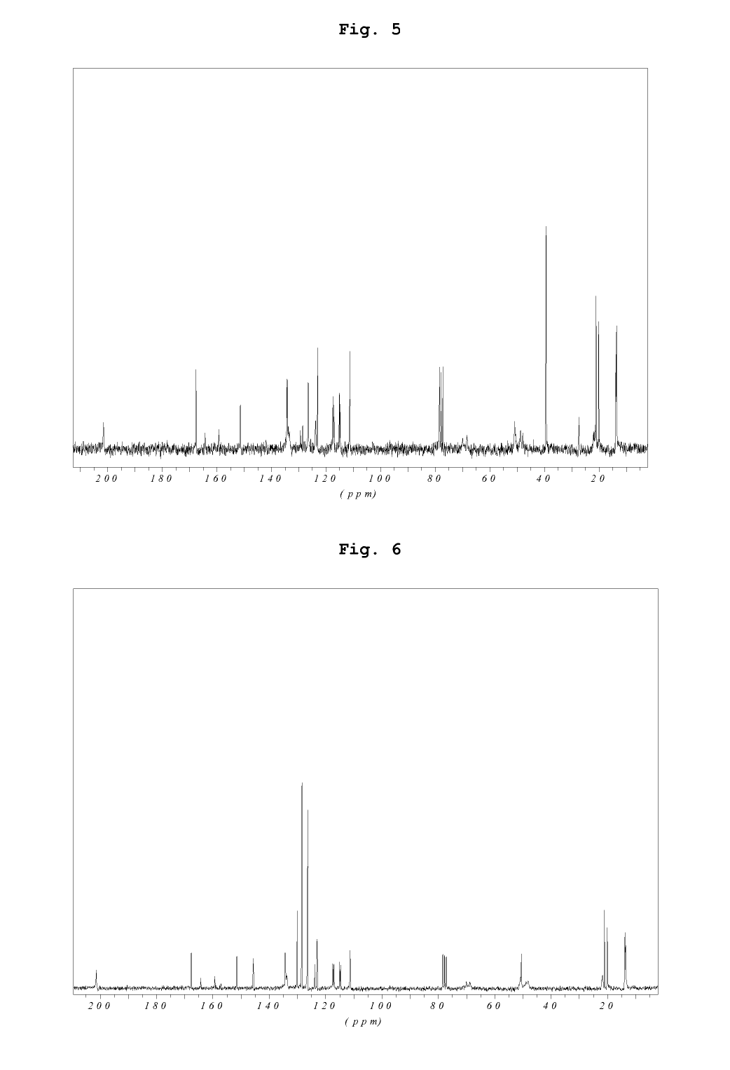

The ¹³C-NMR spectrum of prasugrel ethanesulfonate is shown in FIG. 3, the ¹H-NMR spectrum in FIG. 4.

### Example 10

### Preparation of Prasugrel Mesylate

250 mg prasugrel (free base) were dissolved in 5 ml of methanol and the solution was stirred at room temperature. An equimolar amount of methane-sulfonic acid dissolved in 2 ml methanol was added. The solvent was evaporated in a rotatory evaporator and dried over night in vacuum.

¹³C NMR (CDCl₃, standard: tetramethylsilane; equipment: Bruker DTX 200, 50.3 MHz): δ=13.62 (s), 13.87 (s), 20.12 (s), 21.05(s), 22.07(broad), 39.41 (s), 48.74 (broad), 50.87 (broad), 111.34 (s), 115.01 (d; 12.72 Hz), 117.31 (d; 21.2 Hz), 123.08 (s), 123.87 (s), 126.59 (s),133.38 (broad), 134.65 (d; 9.9 Hz), 151.47 (s), 161.78 (d; 252.9 Hz), 167.6 (s), 201.55 (s) ppm.

The ¹³C-NMR spectrum of prasugrel mesylate is shown in FIG. 5.

### Example 11

### Preparation of Prasugrel-Besylate

250 mg prasugrel (free base) were dissolved in 5 ml of methanol and the solution was stirred at room temperature. An equimolar amount of benzene sulfonic acid dissolved in 2 ml methanol was added. The solvent was evaporated in a rotatory evaporator and dried over night in vacuum.

¹³C NMR (CDCl₃; standard: tetramethylsilane, equipment: Bruker DTX 200, 50.3 MHz): δ=13.59 (s), 13.84 (s), 20.04 (s), 21.05 (s), 21.76 (broad), 48.17 (broad), 50.53 (s), 51.0 (broad), 68.83 (broad), 111.26 (s), 114.85 (d; 14.1 Hz), 117.28 (d; 21.2 Hz), 123.05 (s), 123.87 (s), 126.31 (s), 126.48 (s), 128.29 (s), 130.07 (s), 133.69 (s), 134.28 (d; 8.5 Hz), 145.59 (s), 151.46 (s), 159.30 (d; 250.1 Hz), 167.63 (s), 201.32 (s) ppm.

The ¹³C-NMR spectrum of prasugrel besylate is shown in FIG. 6.

### Example 12

### Preparation of Immediate Release Pharmaceutical Compositions Containing Addition Salts of Prasugrel

1 weight equivalent of prasugrel-besylate was dissolved in methanol, 2 weight equivalents of mannitol were added and stirred until a homogeneous suspension is obtained. The solvent was removed and the residue dried.

Tablets were prepared by dry mixing the ingredients followed by dry compression.

| | amount |
|---|---|
| Ingredient | (wt.-%) |
| prasugrel-besylate/mannitol 1/2 | 30 |
| mannitol | 45 |
| silicon dioxide | 1 |
| microcristalline cellulose | 18 |
| corn starch | 5 |
| stearic acid | 1 |

### Example 13

### Preparation of 10 mg Tablets Containing Prasugrel Hydrochloride, Besylate or Napsylate

### A. Coating Prasugrel Salts on Lactose

### A.1. Coating Prasugrel-Hydrochloride on Lactose (Comparative)

Concentrated hydrochloric acid (37%, d=1.19, 1.054 ml, 13.18 mmol) was added dropwise over 1 minute to a solution of prasugrel (5 g 13.39 mmol) in acetone (50 ml) with stirring at 40° C. The reaction mixture was stirred at the same temperature for 60 minutes. No crystallisation was observed. The volume of the mixture was then reduced to 10 ml and the solution was added dropwise to a suspension of 25 g lactose (Lactopress™anhydrous 265) in 100 ml acetone and stirred for 15 minutes (homogeneous suspension). Acetone was evaporated at 30° C. for 1 hour and at 20° C. over night (rotary vane pump). A free flowing powder was obtained. The prasugrel-HCl lactose premix had a water content of 0.85% (Karl-Fischer titration).

### A.2. Coating Prasugrel-Besylate on Lactose

Benzene sulfonic acid (1.27 g dissolved in 20 ml acetone) was added dropwise to a solution of prasugrel (3 g 8.03 mmol) in acetone (30 ml) and stirred at room temperature for 1 hour. Afterwards 15 g lactose (Lactopress™ anhydrous 265) and 20 ml acetone were added and stirred for additional 15 min. Finally acetone was evaporated at 30° C. for 1 hour and at 20° C. over night (rotary vane pump). A free flowing powder was obtained. The prasugrel-besylate lactose premix had a water content of 1.02% (Karl-Fischer titration).

### A.3. Coating Prasugrel-Napsylate on Lactose

To a solution of prasugrel (3 g 8.03 mmol) in acetone (30 ml) was added dropwise 2-naphthalene sulfonic acid (1.67 g, 8.03 mmol dissolved in 20 ml acetone) and stirred at room temperature for 1 hour. Afterwards 15 g lactose (Lactopress™ anhydrous 265) and 20 ml acetone were added and stirred for additional 15 min. Finally acetone was evaporated at 30° C. for 1 hour and at 20° C. over night (rotary vane pump). A free flowing powder was obtained. The prasugrel-napsylate lactose premix had a water content of 0.77% (Karl-Fischer titration).

### B. Preparation and Stability of Tablets

The premixes described above were used to prepare tablets containing about 10 mg prasugrel.

| | Composition of tablets | | |
|---|---|---|---|
| Component | Tabl. B1*) | Tabl. B2 | Tabl. B3 |
| Prasugrel-HCL (A.1)**) | 50 mg | — | — |
| Prasugrel-besylate (A.2)**) | — | 50 mg | — |
| Prasugrel-napsylat (A.3)**) | — | — | 50 mg |
| Lactose-monohydrate | 70.0 mg | 70.0 mg | 70.0 mg |
| (Tablettose ®) | | | |
| Highly dispersed silica | 2.0 mg | 2.0 mg | 2.0 mg |
| Magnesium stearate | 2.0 mg | 2.0 mg | 2.0 mg |
| Croscarmellose Na | 6.0 mg | 6.0 mg | 6.0 mg |
| (Ac-Di-Sol ®) | | | |
| Total weight | 130 mg | 130 mg | 130 mg |

*)comparative example
**)comprises 10 mg of prasugrel salt and 40 mg of lactose

Tablets were prepared by mixing the ingredients specified in the above table for 5 min. the mixture was sieved through a sieve with 1 mm mesh-size. The granules were pressed to tablets having a diameter of 7 mm using a Kortsch excenter press. The tablets had a hardness of about 60 to 90 N.

The disintegration time (according to Pharm. Eur. 5.0 German version) was 45 sec for tablet B1, 60 sec for tablet B2 and 45 sec for tablet B3.

Tablets were stored for 9 days at room temperature (15-25° C.) in an open container. The tablets containing prasugrel-HCl showed clear discoloration while the tablets containing the sulfonic acid salts of prasugrel showed only negligible discoloration. The tablets containing salts of prasugrel with sulfonic acids thus proved to be more stable than tablets with prasugrel-HCl.

Similar results were obtained with the prasugrel-coated lactose. After 14 days of storage in an open container prasugrel-HCl coated lactose developed a brownish color while the lactose coated with the besylate or napsylate salt remained white.

### Example 14

### Preparation of Crystalline Prasugrel-1,5-Dinapsylate

0.5 g Prasugrel base were suspended in 1.5 ml acetone at room temperature. To the solution 386 mg naphthalene-1,5-disulfonic acid were added under stirring. The solution was sonicated in an ultrasonic bath at room temperature. A few milligrams of Prasugrel-HCl crystals (prepared according to WO2008/000418) were added to the clear solution. A white precipitate was obtained after storing for one night at −20° C. The salt was filtered off and dried at room temperature under vacuum for 6 hours to yield 640 mg. White crystals, melting point 180° C.

1-H NMR (DMSO-d6, standard: tetramethylsilane, equipment: Bruker DTX 200, 200.1 MHz): δ=0.77-1.22 (m, 4H), 1.76-2.00(m, 1H), 2.27 (s, 3H), 2.89-4.7(broad, 6H), 5.97-6.18 (broad, 1H), 6.50-6.65 (broad, 1H), 7.25-7.73 (m, 6H), 7.91 (d, 6.4 Hz, 2H), 8.85(d, 8.6 Hz, 2H), 10.12-11.6 (broad, NH+) ppm

X-Ray (CuK_{α}-radiation, powder diffractometer X′PERT PRO Bragg Brentano flat sample reflection instrument, start position: 3.0 degree 2 Th, end position 65.0 degree 2 Th, step size 0.017 degree 2 Th, PSD length 2.12 degree 2 Th, temperature 25 degree C.):

| Degree 2Th | Rel. Int.*) |
|---|---|
| (±0.2) | (>8%) |
| 8.7 | 9.0 |
| 9.6 | 100.0 |
| 10.6 | 8.8 |
| 14.3 | 21.4 |
| 16.1 | 10.2 |
| 17.5 | 9.8 |
| 17.9 | 8.9 |
| 22.5 | 8.1 |
| 23.8 | 11.7 |

*)relative intensity may change depending on the crystal size and morphology.

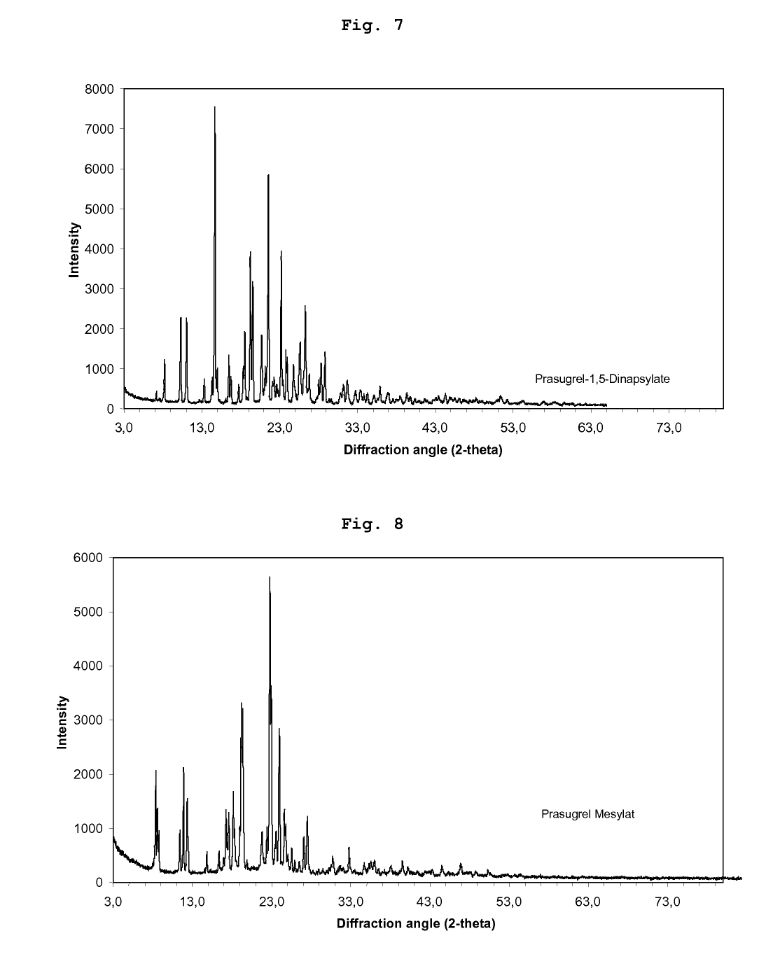

The X-ray diffraction pattern of prasugrel-dinapsylate is shown in FIG. 7.

### Example 15

### Preparation of Crystalline Prasugrel-Mesylate

1 g Prasugrel base was suspended in 4.2 ml acetone at room temperature. To the solution 173.7 μl (257.3 mg) methane sulfonic acid were added under stirring. The solution was sonicated in an ultrasonic bath at room temperature. A few milligrams of Prasugrel-HCl crystals (prepared according to WO2008/000418) were added to the clear solution. A white precipitate was obtained after storage for one night at −20° C. The salt was filtered off and dried at room temperature under vacuum for 6 hours to yield 1004 mg. White crystals, melting point 103° C.

1-H NMR (DMSO-d6, standard: tetramethylsilane, equipment: Bruker DTX 200, 200.1 MHz): δ=0.81-1.2 (m, 4H), 1.81-2.00 (m, 1H), 2.27(s, 3H), 2.30 (s, 3H), 2.92-4.6 (broad, 6H), 5.99-6.16 (broad, 1H), 6.51-6.36 (broad, 1H), 7.32-7.76 (m, 4H), 10.13-11.59 (broad, NH⁺)

X-Ray (CuK_{α}-radiation, powder diffractometer X′PERT PRO Bragg Brentano flat sample reflection instrument, start position: 3.0 degree 2 Th, end position 65.0 degree 2 Th, step size 0.017 degree 2 Th, PSD length 2.12 degree 2 Th, temperature 25 degree C.):

| Degree 2Th | Rel. Int.*) |
|---|---|
| (±0.2) | (>20%) |
| 8.4 | 31.8 |
| 8.6 | 21.1 |
| 11.9 | 35.2 |
| 12.3 | 24.8 |
| 17.2 | 20.9 |
| 17.6 | 20.3 |
| 18.1 | 26.6 |
| 19.2 | 55.4 |
| 19.3 | 52.8 |
| 22.8 | 100.0 |
| 23.0 | 62.4 |
| 24.0 | 48.5 |
| 24.6 | 21.6 |

*)relative intensity may change depending on the crystal size and morphology

The X-ray diffraction pattern of prasugrel-mesylate is shown in FIG. 8.

### Example 16

### Preparation of Crystalline Prasugrel-1-Napsylate

1 g Prasugrel base was suspended in 3 ml acetone at room temperature. To the solution 702 mg naphthalene-1-sulfonic acid were added under stirring. The solution was sonicated in an ultrasonic bath at room temperature. A few milligrams of Prasugrel-HCl crystals (prepared according to WO2008/000418) were added to the clear solution. A white precipitate was obtained after storage for one night at −20° C. The salt was filtered off and dried at room temperature under vacuum for 6 hours to yield 1330 mg. White crystals, melting point 116° C.

1-H NMR (DMSO-d6, standard: tetramethylsilane, equipment: Bruker DTX 200, 200.1 MHz): δ=0.78-1.2 (m, 2H), 1.78-2.00 (m, 1H), 2.27 (s, 3H), 2.87-4.54 (broad, 6H), 5.91-6.23 (broad,1H), 6.47-6.65 (broad, 1H), 7.31-7.77(m, 6H), 7.79-7.98 (m, 3H), 8.76-8.87 (m, 1H), 10.15-11.72 (broad, NH⁺) ppm.

X-Ray (CuK_{α}-radiation, powder diffractometer X′PERT PRO

Bragg Brentano flat sample reflection instrument, start position: 3.0 degree 2 Th, end position 65.0 degree 2 Th, step size 0.017 degree 2 Th, PSD length 2.12 degree 2 Th, temperature 25 degree C.):

| Degree 2Th | Rel. Int.*) |
|---|---|
| (±0.2) | (>8%) |
| 7.3 | 100.0 |
| 9.1 | 11.2 |
| 14.6 | 8.6 |
| 14.8 | 9.6 |
| 15.7 | 8.6 |
| 16.0 | 8.3 |
| 19.0 | 18.2 |
| 20.6 | 9.6 |
| 21.2 | 10.6 |
| 21.6 | 9.9 |
| 23.6 | 14.9 |

*)relative intensity may change depending on the crystal size and morphology

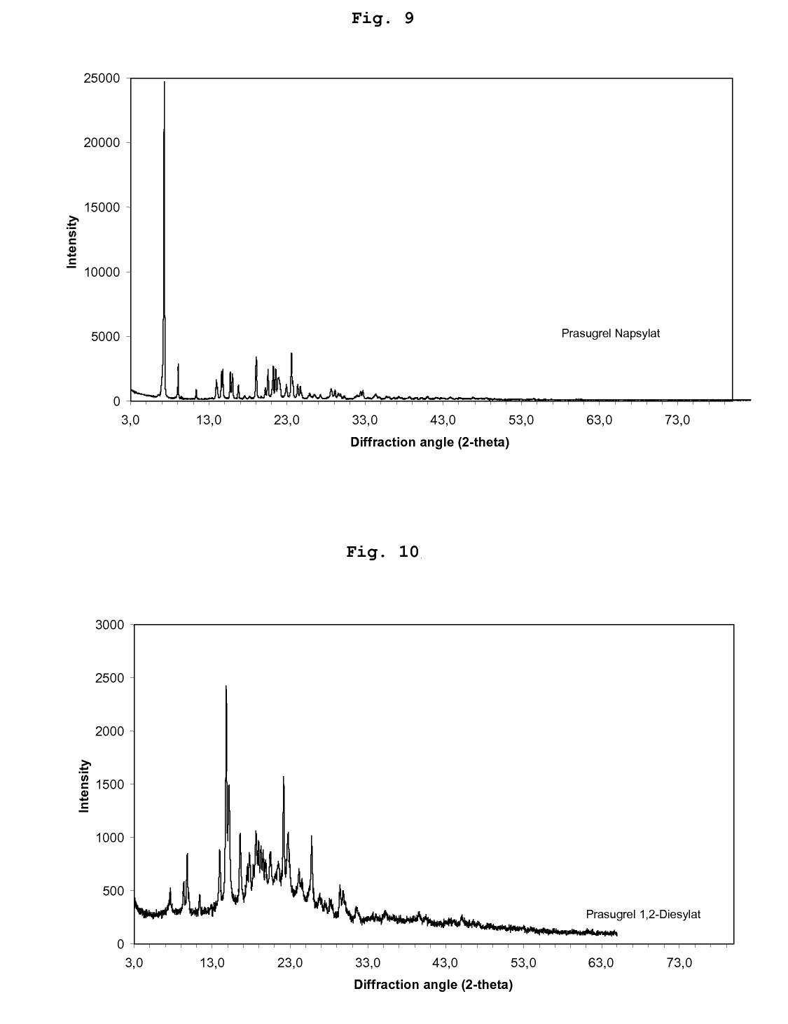

The X-ray diffraction pattern of prasugrel-1-napsylate is shown in FIG. 9.

### Example 17

### Preparation of Crystalline Prasugrel-1,2-Diesylate

0.5 g Prasugrel base was suspended in 1.5 ml acetone at room temperature. To the solution 254.7 mg 1,2-ethane disulfonic acid were added under stirring. A few milligrams of Prasugrel-HCl crystals (prepared according to WO2008/000418) were added to the clear solution. The solution was sonicated in an ultrasonic bath at room temperature. No precipitate was obtained after storage for one night at −20° C. The amount of acetone was reduced (vacuum) and water was added carefully until a white precipitate was noticed, a few crystals of Prasugrel-HCl were additionally added. After storage for one week at −20° C. white crystals were obtained. The salt was filtered off and dried at room temperature under vacuum for 6 hours to yield 80 mg. White crystals, melting point 137° C.

1-H NMR (DMSO-d6, standard: tetramethylsilane, equipment: Bruker DTX 200, 200.1 MHz): δ=0.78-1.23 (m, 4H), 1.80-2.00 (m, 1H), 2.26 (s, 3H), 2.62 (s, 4H), 2.89-4.48 (broad, 6H), 5.98-6.20 (broad, 1H), 6.5-6.65 (broad, 1H), 7.29-7.76 (m, 4H), 10.27-11.59 (broad, NH⁺) ppm

X-Ray (CuK_{α}-radiation, powder diffractometer X′PERT PRO Bragg Brentano flat sample reflection instrument, start position: 3.0 degree 2 Th, end position 65.0 degree 2 Th, step size 0,017 degree 2 Th, PSD length 2.12 degree 2 Th, temperature 25 degree C.):

| Degree 2Th | Rel. Int.*) |
|---|---|
| (±0.2) | (>20%) |
| 9.8 | 26.5 |
| 14.0 | 25.8 |
| 14.8 | 100.0 |
| 15.1 | 53.5 |
| 16.6 | 28.2 |
| 18.6 | 24.7 |
| 22.2 | 50.2 |
| 22.7 | 25.4 |
| 25.8 | 32.0 |

*)relative intensity may change depending on the crystal size and morphology

The X-ray diffraction pattern of prasugrel-diesylate is shown in FIG. 10.

### Example 18

### Preparation of Crystalline Prasugrel-Besylate

1 g Prasugrel base was suspended in 3 ml acetone at room temperature. To the solution 423.9 mg benzene sulfonic acid were added under stirring. A few milligrams of Prasugrel-HCl crystals (prepared according to WO2008/000418) were added to the clear solution. The solution was sonicated in an ultrasonic bath at room temperature. No precipitate was obtained after storage for one night at −20° C. The amount of acetone was reduced (vacuum) and water was added carefully until a white precipitate was noticed, a few crystals of Prasugrel-HCl were additionally added. After storage for one week at −20° C. white crystals were obtained. The salt was filtered off and dried at room temperature under vacuum for 6 hours to yield 1200 mg. White crystals, melting point 168° C.

1-H NMR (DMSO-d6, standard: tetramethylsilane, equipment: Bruker DTX 200, 200.1 MHz): 0.78-1.77(m, 4H), 1.83-2.03 (m, 1H), 2.26 (s, 3H), 2.97-4.2 (broad, 6H), 5.8-6.08 (broad, 1H), 6.54 (broad,1H), 7.22-7.71 (m, 9H) ppm

X-Ray (CuK_{α}-radiation, powder diffractometer X′PERT PRO Bragg Brentano flat sample reflection instrument, start position: 3.0 degree 2 Th, end position 65.0 degree 2 Th, step size 0.017 degree 2 Th, PSD length 2.12 degree 2 Th, temperature 25 degree C.):

| Degree 2Th | Rel. Int.*) |
|---|---|
| (±0.2) | (>20%) |
| 10.3 | 28.3 |
| 11.0 | 28.3 |
| 14.7 | 100.0 |
| 18.5 | 23.4 |
| 19.2 | 50.1 |
| 19.6 | 40.8 |
| 20.7 | 23.1 |
| 21.5 | 77.2 |
| 23.2 | 50.8 |
| 25.6 | 20.7 |
| 26.3 | 32.7 |

*)relative intensity may change depending on the crystal size and morphology

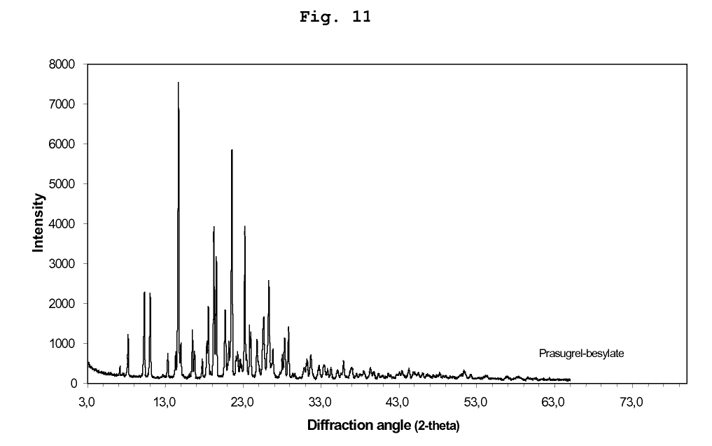

The X-ray diffraction pattern of prasugrel-besylate is shown in FIG. 11.

1. An acid addition salt of 2-acetoxy-5-(α-cyclopropylcarbonyl-2-fluorobenzyl)-4,5,6,7-tetrahydrothieno[3,2-c]pyridine with sulfuric acid or a sulfonic acid.

2. The acid addition salt according to claim 1, wherein said sulfonic acid is an alkylsulfonic acid or an arylsolphonic acid.

3. The acid addition salt of claim 1 or 2, wherein said sulfonic acid is an unsubstituted arylsulfonic acid.

4. The acid addition salt of any one of claims 1 to 3, wherein said sulfonic acid is selected from the group consisting of methanesulfonic acid, ethanesulfonic acid, ethane-1,2-disulfonic acid, 2-hydroxyethanesulfonic acid, benzene sulfonic acid, p-toluenesulfonic acid, 2-naphthalenesulfonic acid, and naphthalene-1,5-disulfonic acid.

5. The acid addition salt of claim 1, wherein said sulfonic acid is selected from the group consisting of benzene sulfonic acid and 2-naphthalenesulfonic acid.

6. The acid addition salt of claim 1, wherein said sulfonic acid is selected from the group consisting of ethane-1,2-disulfonic acid and naphthalene-1,5-disulfonic acid.

7. The acid addition salt of any one of claims 1 to 6, wherein the salt is crystalline.

8. The acid addition salt of claim 7, wherein said sulfonic acid is naphthalene-1,5-disulfonic acid, said salt having an X-ray powder diffraction pattern showing peaks at the following 2-theta values measured by using CuKα-radiation: 8.7; 9.6; 10.6; 14.3; 16.1; 17.5; 17.9; 22.5; 23.8 ±0.2.

9. The acid addition salt of claim 7, wherein said sulfonic acid is methanesulfonic acid, said salt having an X-ray powder diffraction pattern showing peaks at the following 2-theta values measured by using CuKα-radiation: 8.4; 8.6; 11.9; 12.3; 17.2; 17.6; 18.1; 19.2; 19.3; 22.8; 23.0; 24.0; 24.6±0.2.

10. The acid addition salt of claim 7, wherein said sulfonic acid is naphthalene-1-sulfonic acid, said salt having an X-ray powder diffraction pattern showing peaks at the following 2-theta values measured by using CuKα-radiation: 7.3; 9.1; 14.6; 14.8; 15.7; 16.0; 19.0; 20.6; 21.2; 21.6; 23.6±0.2.

11. The acid addition salt of claim 7, wherein said sulfonic acid is ethane-1,2-disulfonic acid, said salt having an X-ray powder diffraction pattern showing peaks at the following 2-theta values measured by using CuKα-radiation: 9.8; 14.0; 14.8; 15.1; 16.6; 18.6; 22.2; 22.7; 25.8±0.2.

12. The acid addition salt of claim 7, wherein said sulfonic acid is benzene sulfonic acid, said salt having an X-ray powder diffraction pattern showing peaks at the following 2-theta values measured by using CuKα-radiation: 10.3; 11.0; 14.7; 18.5; 19.2 19.6; 20.7; 21.5; 23.2; 25.6; 26.3±0.2.

13. A pharmaceutical composition comprising a pharmaceutically effective amount of an acid addition salt according to any one of claims 1 to 12.

14. The pharmaceutical composition of claim 13, further comprising one or more pharmaceutically acceptable excipients.

15. The pharmaceutical composition of claim 13 or 14 for prevention or treatment of thrombosis or a cardiovascular disease in a mammal or human.

16. A method for prevention or treatment of thrombosis or a cardiovascular disease in a mammal or human which comprises administering an effective amount of an acid addition salt according to any one of claims 1 to 12 or of a pharmaceutical composition comprising the same.

17. A process for preparation of the acid addition salt according to any one of claims 1 to 12 which comprises reacting 2-acetoxy-5-(α-cyclopropylcarbonyl-2-fluorobenzyl)-4,5,6,7-tetrahydrothieno[3,2-c]pyridine with a sulfonic acid.
